(19) United States
(12) Patent Application Publication
(10) Pub. No.: US 2019/0276429 A1
(43) Pub. Date: Sep. 12, 2019

(54) 4-(6-(2-(2,4-DIFLUOROPHENYL)-1.1-DIFLUORO-2-HYDROXY-3-(5-MERCAPTO-1H-1,2,4-TRIAZOL-1-YL)PROPYL)PYRIDIN-3-YL)OXY)BENZONITRILE AND PROCESSES OF PREPARATION

(71) Applicant: Dow AgroSciencesLLc, Indianapolis, IN (US)

(72) Inventors: Qiang Yang, Zionsville, IN (US); Nicholas R. Babij, Indianapolis, IN (US); Xiaoyong Li, Midland, MI (US); Sarah Ryan, Indianapolis, IN (US); Yan Han, Zionsville, IN (US)

(21) Appl. No.: 16/462,183

(22) PCT Filed: Nov. 17, 2017
(86) PCT No.: PCT/US2017/062151

§ 371 Date: May 17, 2019

Related U.S. Application Data
(60) Provisional application No. 62/423,845, filed on Nov. 18, 2016.

(51) Int. Cl. C07D 401/06

(52) U.S. Cl. CPC C07D 401/06

(57) ABSTRACT

Provided herein is a process for the preparation of 4-(6-(2-(2,4-difluorophenyl)-1.1-difluoro-2-hydroxy-3-(5-mercapto-1H-1,2,4-triazol-1-yl)propyl)pyridin-3-yl)oxy)benzonitrile.

### CROSS-REFERENCE TO RELATED APPLICATIONS

The present application claims priority under 35 U.S.C. § 119(e) to U.S. provisional patent application, U.S. Ser. No. 62/423,845, filed Nov. 18, 2016, the entire contents of which is incorporated herein by reference.

### FIELD

Provided herein is 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile and processes of preparation.

### BACKGROUND

U.S. Patent Application Ser. No. 62/163,106 describes inter alia certain metalloenzyme inhibitor compounds and their use as fungicides. The disclosure of this application is expressly incorporated by reference herein. This patent applications describes various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) and processes for its preparation. In one embodiment, provided herein, is a process for the preparation of the compound of the Formula I:

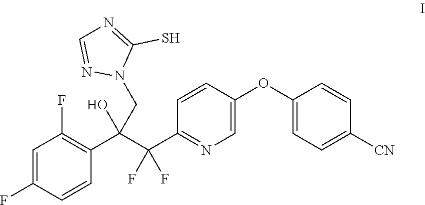 [SMILES: N#Cc1ccc(Oc2ccc(C(F)(F)C(O)(Cn3ncnc3S)c3ccc(F)cc3F)nc2)cc1]

which comprises contacting a compound of Formula II with an alkanedithiol, a copper salt and a base,

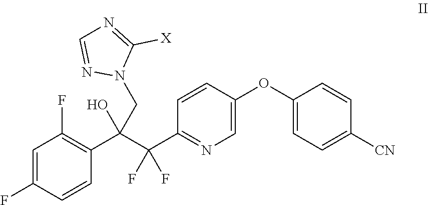

wherein X=Cl, Br or I.

In another embodiment, the compound of Formula II may be prepared by contacting a compound of Formula III with a halogenating agent and a free radical initiator compound.

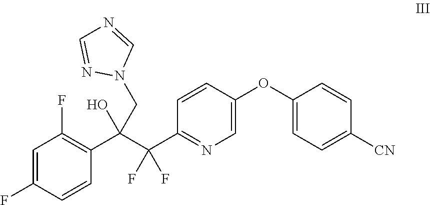 [SMILES: N#Cc1ccc(Oc2ccc(C(F)(F)C(O)(Cn3cncn3)c3ccc(F)cc3F)nc2)cc1]

Another aspect of the present disclosure is the novel intermediate produced in the present process, viz., a compound consisting of:

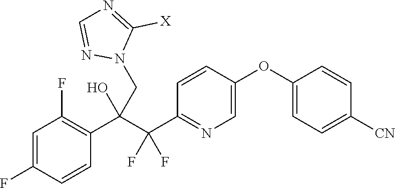

wherein X=Cl, Br or I.

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I-III are read as also including optical isomers and salts. Specifically, when compounds of Formula I-III contain a chiral carbon, it is understood that such compounds include optical isomers and racemates thereof. Exemplary salts may include: hydrochloride salts, hydrobromide salts, hydroiodide salts, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule. For example, the chemical structures of Formulas I and Ia are tautomeric forms of the same molecule.

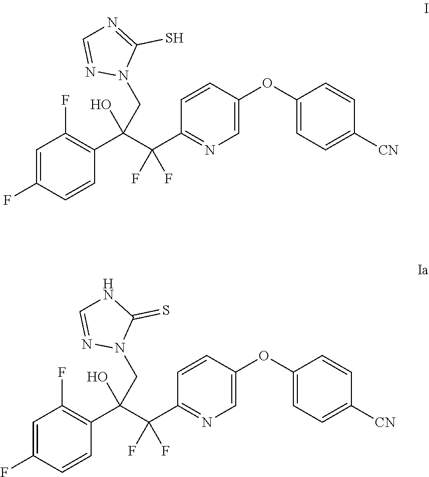 [SMILES: N#Cc1ccc(Oc2ccc(C(F)(F)C(O)(Cn3ncnc3S)c3ccc(F)cc3F)nc2)cc1 ; N#Cc1ccc(Oc2ccc(C(F)(F)C(O)(Cn3nc[nH]c3=S)c3ccc(F)cc3F)nc2)cc1]

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-Difluorophenyl)-1,1-difluoro-2-hydroxy-3-(5-mercapto-1H-1,2,4-triazol-1-yl)propyl)pyridin-3-yl)oxy)benzonitrile (I) is provided herein and may be prepared from 4-((6-(2-(2,4-difluorophenyl)-1,1-difluoro-2-hydroxy-3-(5-halo-1H-1,2,4-triazol-1-yl)propyl)pyridin-3-yl)oxy)benzonitrile (II) as shown in Example 1.

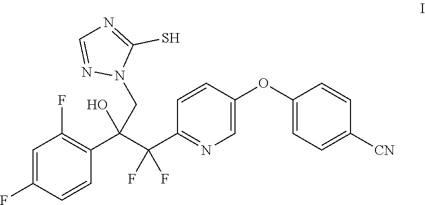 [SMILES: N#Cc1ccc(Oc2ccc(C(F)(F)C(O)(Cn3ncnc3S)c3ccc(F)cc3F)nc2)cc1]

### Example 1: Preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I)

### Method 1A: Preparation of the compound of Formula I from 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa)

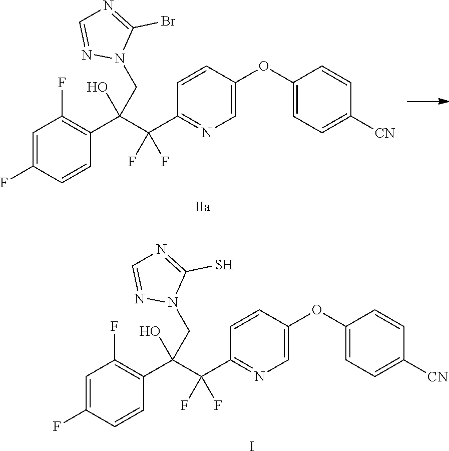

To a 100-mL reactor was charged 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa) (1 g, 1.824 mmol), ethane-1,2-dithiol (0.306 mL, 3.65 mmol), CuSO₄*5H₂O (45.5 mg, 0.182 mmol), Cs₂CO₃ (1.19 g, 3.65 mmol), and DMSO (5 mL). The mixture was heated at 100° C. for 4 h, at which point HPLC analysis indicated that the reaction was complete. The reaction mixture was cooled to 20° C. and diluted with water (20 mL). The mixture was extracted with EtOAc (2×20 mL) (solid precipitation observed) and the combined organics were concentrated to dryness. The residue was purified by silica gel column chromatography (40 g silica, eluent: 0-60% EtOAc/hexanes over 5 column volumes, hold for 5 column volumes). The pure fractions were concentrated to afford the desired product (I) as a light brown solid (620 mg, 68% yield). ¹H NMR (400 MHz, DMSO-d₆) δ 13.59 (s, 1H), 8.46 (d, J=2.7 Hz, 1H), 8.18 (s, 1H), 7.95-7.85 (m, 2H), 7.71 (dd, J=8.7, 2.8 Hz, 1H), 7.63 (d, J=8.6 Hz, 1H), 7.37 (td, J=9.0, 6.8 Hz, 1H), 7.25-7.19 (m, 2H), 7.19-7.11 (m, 1H), 6.95 (td, J=8.5, 2.6 Hz, 1H), 6.42 (s, 1H), 5.15-4.99 (m, 2H). ESIMS m/z 501.9 ([M+H]+).

### Method 1B: Preparation of the compound of Formula I from 4-((6-(3-(5-iodo-1H-1,2,4-triazol-1-yl)-2-(2,4-difluorophenyl)-1,1-difluoro-2-hydroxypropyl)pyridin-3-yl)oxy)benzonitrile (IIb)

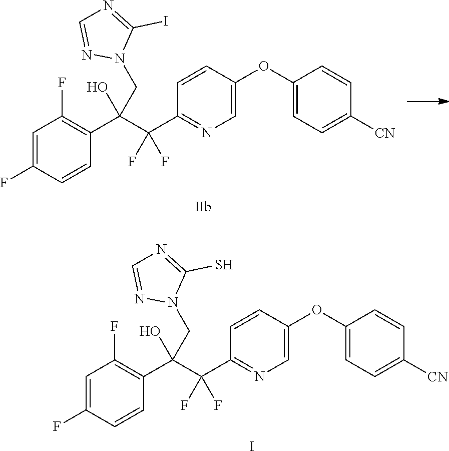

To a 100-mL reactor was charged 4-((6-(3-(5-iodo-1H-1,2,4-triazol-1-yl)-2-(2,4-difluorophenyl)-1,1-difluoro-2-hydroxypropyl)pyridin-3-yl)oxy)benzonitrile (IIb) (1 g, 1.68 mmol), ethane-1,2-dithiol (0.282 mL, 3.36 mmol), CuSO₄*5H₂O (41.9 mg, 0.168 mmol), Cs₂CO₃ (1.10 g, 3.36 mmol), and DMSO (5 mL). The mixture was heated at 100° C. for 4 h, after which the reaction mixture was cooled to 20° C. and diluted with water (20 mL). The mixture was extracted with EtOAc (2×20 mL) and the combined organics were concentrated to dryness. The residue was purified by silica gel column chromatography (40 g silica, eluent: 0-60% EtOAc/hexanes over 5 column volumes, hold for 5 column volumes). The pure fractions were concentrated to afford the desired product as a light brown solid (I) (380 mg, 45% yield). Analytical data was consistent with that of previously isolated sample from Method 1A.

Suitable solvents for use in this process step may include acetonitrile (MeCN), N,N-dimethylformamide (DMF), N,N-dimethylacetamide (DMA), dimethyl sulfoxide (DMSO), sulfolane, and N-methyl-2-pyrrolidone (NMP), and mixtures of these solvents with water.

Suitable bases for use in this process step may include metal carbonates, metal alkoxides, metal hydroxides, and metal phosphates such as, for example, potassium carbonate, sodium carbonate, cesium carbonate, sodium methoxide, potassium methoxide, sodium hydroxide, potassium hydroxide, sodium phosphate, and potassium phosphate.

Suitable copper salts for use in this process step may include, for example, CuSO₄, CuCl₂, CuBr₂, Cu(acac)₂, CuO, Cu(OH)₂, Cu(OAc)₂, CuBr, CuI, and Cu₂O.

Alkanedithiols that may be used in this process step include, for example, ethane-1,2-dithiol, butane-1,4-dithiol and other similar alkanedithiols.

This process step may be conducted at temperatures from about 25° C. to about 200° C., or from about 50° C. to about 150° C. 4-((6-(2-(2,4-Difluorophenyl)-1,1-difluoro-2-hydroxy-3-(5-halo-1H-1,2,4-triazol-1-yl)propyl)pyridin-3-yl)oxy)benzonitrile (II; X=Br or I) may be prepared from 4-((6-(2-(2,4-difluorophenyl)-1,1-difluoro-2-hydroxy-3-(1H-1,2,4-triazol-1-yl)propyl)pyridin-3-yl)oxy)benzonitrile (III) as shown in Examples 2 and 3.

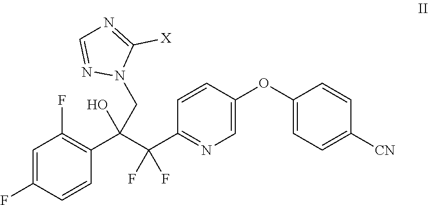

### Example 2: Preparation of 4-((6-(2-(2,4-difluorophenyl)-1,1-difluoro-2-hydroxy-3-(5-bromo-1H-1,2,4-triazol-1-yl)propyl)pyridin-3-yl)oxy)benzonitrile (IIa) from the compound of Formula III

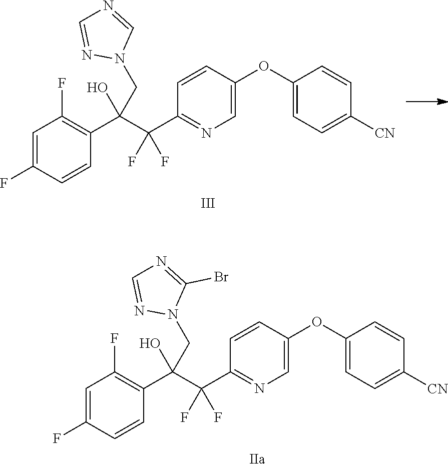

### Method 2A: Bromination with NBS and Benzoyl Peroxide in Acetonitrile

To a 100-mL 3-neck round bottom flask was charged 4-((6-(2-(2,4-difluorophenyl)-1,1-difluoro-2-hydroxy-3-(1H-1,2,4-triazol-1-yl)propyl)pyridin-3-yl)oxy)benzonitrile (III) (3.00 g, 6.39 mmol), N-bromosuccinimide (1.706 g, 9.59 mmol), benzoic peroxyanhydride (benzoyl peroxide; 0.310 g, 1.278 mmol), and MeCN (15 mL). The reaction was heated at 80° C. for 18 h, at which point HPLC analysis indicated that the reaction was complete. The reaction was cooled to 20° C. and purified directly by column chromatography (80 g silica, eluent: EtOAc/hexanes 0-50% over 6 volumes, hold for 5 volumes). The pure fractions were concentrated to dryness to afford a white solid, which was suspended in MTBE (10 mL) and filtered. The solid was dried to afford the desired product (IIa) as a white solid (2.0 g, 57% yield). mp: 170-172° C. ¹H NMR (400 MHz, DMSO-d₆) δ 8.51 (d, J=2.7 Hz, 1H), 8.00-7.88 (m, 2H), 7.84 (s, 1H), 7.72 (dd, J=8.7, 2.8 Hz, 1H), 7.61 (d, J=8.7 Hz, 1H), 7.34 (td, J=9.0, 6.6 Hz, 1H), 7.27-7.09 (m, 3H), 6.98 (td, J=8.5, 2.6 Hz, 1H), 6.88 (s, 1H), 5.20 (d, J=14.5 Hz, 1H), 4.91 (d, J=14.6 Hz, 1H). ¹³C NMR (101 MHz, DMSO-d₆) δ 164.23 (d, J=12.7 Hz), 161.71 (t, J=11.9 Hz), 160.22, 159.17 (d, J=12.4 Hz), 153.10, 152.67, 147.66 (t, J=27.9 Hz), 141.37, 135.36, 132.46, 132.28 (dd, J=9.6, 5.1 Hz), 128.06, 124.66 (t, J=4.6 Hz), 120.74 (dd, J=12.1, 4.0 Hz), 119.51 (t, J=256.5 Hz), 119.35, 118.92, 111.15 (dd, J=20.8, 3.1 Hz), 107.02, 104.51 (dd, J=29.3, 25.7 Hz), 78.12 (td, J=28.3, 4.7 Hz), 51.54 (d, J=9.3 Hz). ESIMS m/z 549.9 ([M+H]+).

### Method 2B: Bromination with NBS and Benzoyl Peroxide in EtOAc

To a mixture of 4-((6-(2-(2,4-difluorophenyl)-1,1-difluoro-2-hydroxy-3-(1H-1,2,4-triazol-1-yl)propyl)pyridin-3-yl)oxy)benzonitrile (0.5 g, 1.065 mmol), NBS (0.284 g, 1.598 mmol), and benzoyl peroxide (0.052 g, 0.213 mmol) was added EtOAc (2.5 mL) and the reaction was heated at 75° C. for 4 h. Saturated sodium thiosulfate solution was added and the layers were separated. The organic layer was concentrated to an oil and purified by normal phase chromatography (0-50% EtOAc/hexanes). Product containing fractions were collected and concentrated giving 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa) (460 mg, 0.839 mmol, 79% yield).

### Method 2C: Bromination with NBS and AIBN in MeCN

To a mixture of 4-((6-(2-(2,4-difluorophenyl)-1,1-difluoro-2-hydroxy-3-(1H-1,2,4-triazol-1-yl)propyl)pyridin-3-yl)oxy)benzonitrile (0.5 g, 1.065 mmol), NBS (0.284 g, 1.598 mmol), and AIBN (0.035 g, 0.213 mmol) was added acetonitrile (2.5 mL) and the reaction was heated at 75° C. for 6 h. The reaction was diluted with DCM and loaded directly onto a silica gel cartridge and purified by normal phase chromatography (0-50% EtOAc/hexanes). Product containing fractions were collected and concentrated giving 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa) (520 mg, 0.854 mmol, 80% yield).

### Method 2D: Bromination with NBS and AIBN in EtOAc

To a mixture of 4-((6-(2-(2,4-difluorophenyl)-1,1-difluoro-2-hydroxy-3-(1H-1,2,4-triazol-1-yl)propyl)pyridin-3-yl)oxy)benzonitrile (0.5 g, 1.065 mmol), NBS (0.284 g, 1.598 mmol), and AIBN (0.035 g, 0.213 mmol) was added ethyl acetate (2.5 mL) and the reaction was heated at 75° C. for 4 h. Saturated sodium thiosulfate solution was added and the layers were separated. The organic layer was concentrated to an oil and purified by normal phase chromatography (0-50% EtOAc/hexanes). Product containing fractions were collected and concentrated giving 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa) (380 mg, 0.485 mmol, 45.5% yield).

### Method 2E: Bromination with DBDMH and Benzoyl Peroxide in EtOAc

To a vial was added 4-((6-(2-(2,4-difluorophenyl)-1,1-difluoro-2-hydroxy-3-(1H-1,2,4-triazol-1-yl)propyl)pyridin-3-yl)oxy)benzonitrile (0.5 g, 1.065 mmol), benzoic peroxyanhydride (0.052 g, 0.213 mmol), 1,3-dibromo-5,5-dimethylimidazolidine-2,4-dione (DBDMH); 0.305 g, 1.065 mmol), and ethyl acetate (2.5 mL) and the reaction was heated at 75° C. for 3 h. The reaction was partitioned between ethyl acetate and saturated sodium thiosulfate solution. The organic layer was concentrated and purified by normal phase chromatography (0-50% EtOAc/hexanes) giving 4-((6-(3-(5-bromo-1H-1,2,4-triazol-1-yl)-2-(2,4-difluorophenyl)-1,1-difluoro-2-hydroxypropyl)pyridin-3-yl)oxy)benzonitrile (IIa) (450 mg, 0.739 mmol, 69.3% yield) as a pale yellow foam.

### Example 3: Preparation of 4-((6-(2-(2,4-difluorophenyl)-1,1-difluoro-2-hydroxy-3-(5-iodo-1H-1,2,4-triazol-1-yl)propyl)pyridin-3-yl)oxy)benzonitrile (IIb)

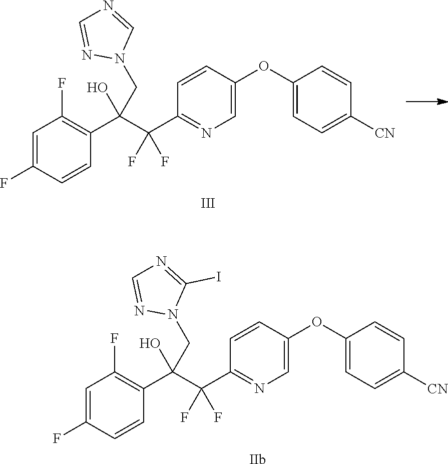

To a 100-mL 3-neck round bottom flask was charged 4-((6-(2-(2,4-difluorophenyl)-1,1-difluoro-2-hydroxy-3-(1H-1,2,4-triazol-1-yl)propyl)pyridin-3-yl)oxy)benzonitrile (III) (2.00 g, 4.26 mmol), N-iodosuccinimide (1.438 g, 6.39 mmol), benzoic peroxyanhydride (benzoyl peroxide; 0.206 g, 0.852 mmol), and MeCN (15 mL). The reaction was heated at 80° C. for 40 h, at which point HPLC analysis indicated that the reaction was complete. The reaction was cooled to 20° C. and purified directly by column chromatography (80 g silica, eluent: EtOAc/hexanes 0-50% over 6 volumes, hold for 5 volumes). The pure fractions were concentrated to dryness to afford a white solid, which was suspended in MTBE (10 mL) and filtered. The solid was dried to afford the desired product (IIb) as an off-white solid (1.58 g, 61% yield). mp: 188-190° C. ¹H NMR (400 MHz, DMSO-d₆) δ 8.54 (d, J=2.7 Hz, 1H), 8.00-7.87 (m, 2H), 7.81 (s, 1H), 7.74 (dd, J=8.7, 2.8 Hz, 1H), 7.64 (d, J=8.7 Hz, 1H), 7.38 (td, J=9.0, 6.7 Hz, 1H), 7.28-7.22 (m, 2H), 7.18 (ddd, J=12.0, 9.1, 2.7 Hz, 1H), 7.00 (td, J=8.5, 2.6 Hz, 1H), 6.82 (s, 1H), 5.21 (d, J=14.4 Hz, 1H), 4.90 (d, J=14.5 Hz, 1H). ¹³C NMR (101 MHz, DMSO-d₆) δ 164.18 (d, J=12.7 Hz), 161.68 (dd, J=12.7, 9.1 Hz), 160.24, 159.15 (d, J=12.3 Hz), 154.37, 153.10, 147.76 (t, J=27.9 Hz), 141.39, 135.38, 132.33 (dd, J=9.6, 5.4 Hz), 128.12, 124.77 (t, J=3.5 Hz), 120.94 (dd, J=36.0, 3.5 Hz), 119.50 (t, J=253 Hz), 119.36, 111.12 (dd, J=21.5, 2.5 Hz), 107.71, 107.00, 104.51 (dd, J=29.0, 25.9 Hz), 78.22 (td, J=27.9, 4.3 Hz), 52.45 (d, J=8.8 Hz). ESIMS m/z 595.9 ([M+H]+).

Halogenating reagents that may be used in this process step include N-bromosuccinimide (NBS), N-chlorosuccinimide (NCS), N-iodosuccinimide (NIS), chlorine (Cl₂), bromine (Br₂), iodine (I₂), 1,3-dibromo-5,5-dimethylhydantoin, 1,3-dichloro-5,5-dimethylhydantoin, 1,3-diiodo-5,5-dimethylhydantoin, mixed halogens such as BrCl, and ICl, and mixtures of halide salts and oxidizing agents such as, for example, NaBr/H₂O₂.

Free radical initiator compounds that may be used in this step include, but are not limited to, benzoyl peroxide, and azobisisobutyronitrile (AIBN).

Suitable solvents for use in this process step may include acetonitrile (MeCN), N,N-dimethylformamide (DMF), N-methyl-2-pyrrolidone (NMP), toluene, ethyl acetate, and mixtures thereof.

This process step may be conducted at temperatures from about 25° C. to about 150° C., or from about 50° C. to about 125° C.

1. A method of making a compound of Formula I 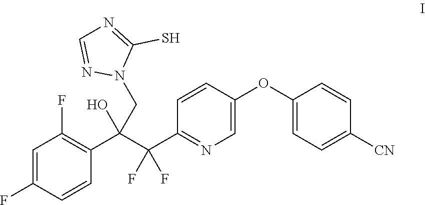 [SMILES: N#Cc1ccc(Oc2ccc(C(F)(F)C(O)(Cn3ncnc3S)c3ccc(F)cc3F)nc2)cc1]
comprising the step of contacting a compound of Formula II 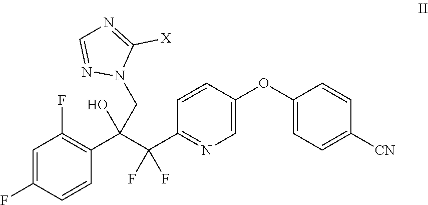
wherein X is Cl, Br or I;
with an alkanedithiol, a copper salt and a base.

2. The method of claim 1, wherein the copper salt may be selected from the group including CuSO₄, CuCl₂, CuBr₂, Cu(acac)₂, Cu(OAc)₂, Cu(OH)₂, CuO, and mixtures thereof.

3. The method of claim 1, wherein the base is selected from the group including a metal carbonate, a metal alkoxide, a metal phosphate, and a metal hydroxide.

4. The method of claim 1, wherein the base is selected from the group including potassium carbonate, sodium carbonate, and cesium carbonate.

5. The method of claim 1 further comprising a solvent selected from the group including acetonitrile, N,N-dimethylacetamide, dimethylsulfoxide, N,N-dimethylformamide, N-methyl-2-pyrrolidone, sulfolane, and mixtures thereof, and mixtures of any of these solvents with water.

6. The method of claim 1 wherein the contacting is carried out between about 25° C. and about 200° C.

7. The method of claim 1 wherein the contacting is carried out between about 50° C. and about 150° C.

8. The method of claim 1, further comprising the step of contacting a compound of Formula III 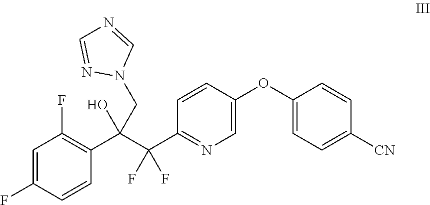 [SMILES: N#Cc1ccc(Oc2ccc(C(F)(F)C(O)(Cn3cncn3)c3ccc(F)cc3F)nc2)cc1]
with a halogenating agent and a free radical initiator compound to prepare the compound of Formula II, wherein X is a Cl, Br, or I.

9. The method of claim 8, wherein the halogenating agent may be selected from the group including NBS, NCS, NIS, Br₂, Cl₂, I₂, 1,3-dibromo-5,5-dimethylhydantoin, 1,3-dichloro-5,5-dimethylhydantoin, 1,3-diiodo-5,5-dimethylhydantoin, and NaBr/H₂O₂.

10. The method of claim 8, wherein the free radical initiator compound may be selected from benzoyl peroxide or azobisisobutyronitrile (AIBN).

11. The method of claim 8 further comprising a solvent selected from the group including acetonitrile, N,N-dimethylformamide, N-methyl-2-pyrrolidone, toluene, and ethyl acetate.

12. The method of claim 8 wherein the contacting is carried out between about 25° C. and about 150° C.

13. The method of claim 8 wherein the contacting is carried out between about 50° C. and about 125° C.

14. A compound consisting of: 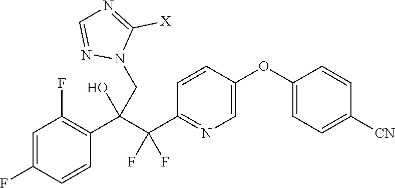
wherein X=Cl, Br or I.
